(19) United States
(12) Patent Application Publication
(10) Pub. No.: US 2020/0278332 A1
(43) Pub. Date: Sep. 3, 2020

(54) METHOD AND TEST DEVICE FOR TESTING A GAS SENSOR, AND SYSTEM CONSISTING OF A GAS SENSOR AND A TEST DEVICE

(71) Applicant: DRÄGER SAFETY AG & CO. KGAA, Lübeck (DE)

(72) Inventors: Mladen SCHLICHTE, Lübeck (DE); Rigobert CHRZAN, Bad Oldesloe (DE); Peter TSCHUNCKY, Lübeck (DE)

(73) Assignee: DRÄGER SAFETY AG & CO. KGAA, Lübeck (DE)

(21) Appl. No.: 16/761,113

(22) PCT Filed: Oct. 5, 2018
(86) PCT No.: PCT/EP2018/077083

§ 371 Date: May 1, 2020

(30) Foreign Application Priority Data
Nov. 3, 2017 (DE) 10 2017 010 222.8

(51) Int. Cl. G01N 33/00

(52) U.S. Cl. CPC G01N 33/007; G01N 2033/0072

(57) ABSTRACT

The invention relates to a method for testing a gas sensor by exposure to a test gas by a test device. The invention also relates to a test device for testing a gas sensor by applying a test gas. The invention also relates to a system comprising a gas sensor and a test device for testing the gas sensor by applying a test gas.

### BACKGROUND OF THE INVENTION

The present invention relates to a method for testing a gas sensor by applying a test gas to a test device. Furthermore, the present invention relates to a test device for testing a gas sensor by exposure to a test gas, and to a system comprising a gas sensor and a test device for testing the gas sensor by exposure to a test gas.

It is generally known to use gas sensors for monitoring atmospheric environments. Gas sensors of this type can be installed, for example, in detector devices used in workplace environments in order to determine concentrations of potentially harmful gases in the ambient air and, if appropriate, to indicate an excessive concentration of such a harmful gas. In order to be able to ensure the function of a gas sensor of such a detector device at all times, it is also known to regularly test the built-in gas sensor. According to the prior art, a test gas from a gas reservoir is provided for such a test or is produced by a gas generator and is supplied to the gas sensor in a substantially constant concentration.

It has turned out to be problematic that this simple, constant supply of test gas may not always accurately reflect the functional status of the installed gas sensor. For example, environmental influences, such as a changed temperature, a changed air pressure, a changed air humidity or wind influences, can influence a measurement of the gas sensor with regard to the test gas provided. It is often not possible to make more than a limited assessment of the sensor operation, i.e. whether the sensor is providing a measurement signal or not. In particular, qualitative and/or quantitative statements about a functional status of the installed gas sensor are usually not possible.

Proceeding from this, an object of the invention is to at least partially overcome these disadvantages of methods for testing a gas sensor, of test devices for testing a gas sensor and of systems comprising a gas sensor and a test device. It is therefore the object of the present invention to provide a method for testing a gas sensor, a test device for testing a gas sensor, and a system comprising a gas sensor and a test device, which make it possible, in a particularly simple and cost-effective manner, to give the most accurate possible information about a functional status of a gas sensor to be installed in a gas detector. Specifically quantitative and qualitative evaluations of the functional status of the installed gas sensor are made possible.

### BRIEF DESCRIPTION OF THE DRAWINGS

FIG. 1 shows a method according to an embodiment of the invention;

FIG. 2 shows a system according to an embodiment of the invention;

FIG. 3 shows a first embodiment of a test sequence according to the invention; and

FIG. 4 shows a second embodiment of a test sequence according to the invention.

### DISCLOSURE OF THE INVENTION

The above object is achieved by a method for testing a gas sensor with the features of independent claim 1. Furthermore, the object is achieved by a test device for testing a gas sensor with the features of independent claim 12. In addition, the object is achieved by a system comprising a Gas sensor and a test device with the features of the independent claim 14. Further features and details of the invention emerge from the subclaims, the description and the drawings. Features and details that are described in connection with the method according to the invention apply, of course, also in connection with the test device according to the invention and the system according to the invention and vice versa, so that the various aspects of the invention are always or can be mutually referred to.

According to a first aspect of the invention, the object is achieved by a method for testing a gas sensor by applying a test gas to a test device. A method according to an embodiment of the invention is characterized by the following steps:

a) establishing a test sequence, the test sequence comprising at least one test duration and a test flow of test gas during the test duration, the test flow further comprising at least two different test gas concentrations over the test duration,

b) providing and supplying the test gas to the gas sensor on the basis of the test sequence defined in step a),

c) determining a sensor response of the gas sensor to the test flow of test gas supplied in step b), and

d) evaluating the sensor response determined in step c) to complete the test of the gas sensor.

Gas sensors are fundamentally known and are used, for example, in gas detectors for monitoring environments, for example, work environments. By means of a method according to an embodiment of the invention, by testing the gas sensor, the functional status of a gas sensor installed in this way can be ensured by exposure to a test gas by a test device. In other words, the test gas is provided by the test device, for example from a reservoir or produced by a gas generator, and is supplied to the gas sensor. A measurement of the gas sensor, in which the supplied test gas is recognized and measured by the test device in order to complete the test of the gas sensor.

A test sequence according to an embodiment of the invention is defined in detail in a first step a). Such a test sequence is characterized by a test duration, a certain test flow of test gas being simultaneously defined as the test sequence during the test duration. Preferably, this test flow has at least two different test gas concentrations over the test duration. In other words, the test sequence thus describes a gas flow which lasts over a test period and has a change in a concentration of the test gas provided during this test period. The test gas provided is therefore subject to a time-dependent change with regard to a concentration of test gas provided. The change in concentration can be generated by changing the amount of test gas provided per unit of time.

In the next step b), according to a method according to an embodiment of the invention, the test gas is provided and supplied to the gas sensor, this provision and supply being carried out on the basis of the test sequence defined in step a). In other words, in step b) the gas sensor is successively subjected to a test flow of test gas with at least two different test gas concentrations over the test duration. The gas sensor, which can be in its normal operating mode and thus constantly monitors and measures an ambient gas, can therefore preferably provide different sensor responses, which in the best case are correlated with the different test gas concentrations.

In the following step c), these sensor responses of the gas sensor to the test flow of test gas supplied in step b) are determined. This determination can be provided, for example, by simply tapping an electrical signal that the gas sensor outputs as a measurement signal. In other words, the gas sensor is read out in step c). After step c) has been carried out, the information is then available on how the measurement results of the gas sensor have changed during the supply of the test gas during the test sequence.

The last step d) of a method according to an embodiment of the invention includes an evaluation of the sensor response determined in step c). This evaluation can include, for example, a pure detection of whether the sensor response contains a signal shape that at least essentially indicates the test gas supplied according to the test sequence. Depending on the result of this evaluation, information can thus be provided at the end of the test of the gas sensor as to whether the gas sensor is functioning within its performance parameters or not.

In summary, a test of a gas sensor can be improved by a method according to the invention. By providing and supplying the test gas to the gas sensor based on a test sequence with at least two different test gas concentrations during a test period, environmental influences can be filtered out from the sensor response and a determination of the functional status of the gas sensor can be improved. The method provides the ability to assess the influences of environmental influences, such as air pressure, temperature or the like, from the sensor response. This can be provided since these influences are relatively constant over the test duration, and because the test sequence comprises two different test gas concentrations. In this way, qualitative and quantitative statements about a state of the gas sensor can also be made possible.

A method according to an embodiment of the invention can preferably be further developed in such a way that in step a) at least one of the at least two different test gas concentrations is defined with a time-variable test gas concentration profile. The profile enables that at least one of the two test gas concentrations can be provided not only as a constant test gas concentration, but also as a test gas concentration that changes continuously over time. By ascertaining a sensor response to this time-variable, continuously changing, test gas concentration profile, the accuracy of the determination of the functional status of the gas sensor can be further increased.

According to a preferred embodiment of a method according to the invention, the test gas concentration profile defined in step a) has at least one of the following forms:

Triangle

Ramp

Rectangle

Stairs

Trapezoid

Sine

This list is not exhaustive, so that, if technically meaningful and possible, other forms may also be identified by the test gas concentration profile defined in step a). A diverse array of test gas concentration profiles can be provided in this way.

Furthermore, it can be provided in embodiments of the method according to the invention that in step a) the test sequence comprises gas flow interruptions, wherein the supply of test gas to the gas sensor is stopped. When evaluating the sensor response, it can be verified whether these interruptions in the gas flow can also be detected. This alone can further increase the accuracy when testing the gas sensor using a method according to the invention. In addition, these gas flow pauses allow measurement of the background, which is measured by the gas sensor without the addition of test gas, even during an ongoing test sequence. In this way, too, an overall accuracy in determining a functional status of a gas sensor can be further increased by a method according to the invention.

A method according to an embodiment of the invention can also be designed such that in step a) the test sequence is defined with two or more test sequence sections. The test sequence sections are arranged one after the other over the test duration. Each test sequence section gas a duration and at least one test section flow of test gas during the test section duration. The test section flow also has at least two different test gas concentrations over the test section duration. In other words, the test sequence sections are essentially designed like a test sequence described above. By having several such test sequence sections within a test sequence, even more different measurements of the gas sensor can be carried out at different test gas concentrations during a test sequence. As a result of this increase in the individual measurements, an overall accuracy in determining a functional status of the gas sensor can be further increased by a method according to the invention.

A method according to an embodiment of the invention can preferably be designed that in step a) the two or more test sequence sections are defined separately from one another by gas flow interruptions. As already described above, gas flow interruptions can be used, on the one hand to be detected by the user and on the other hand to be able to provide a background measurement during an ongoing test sequence. In an embodiment, such a gas flow interruption can additionally be used to determine or recognize an exact separation of the two test sequence sections in the determined sensor response of the gas sensor. This also makes it possible to increase the accuracy when determining a functional status of the gas sensor being checked.

A method according to a preferred embodiment of the invention can be designed that in step a) the two or more test sequence sections are defined identically. In other words, it can be provided that two or more identical test sequence sections with identical test section durations and test section flows are used. An ideally functioning gas sensor will therefore deliver the same sensor response for each of the test sequence sections used. By comparing the determined sensor responses for the different test sequence sections, a determination of a functional status of the gas sensor can also be carried out more precisely.

In addition, it can be provided in an embodiment of a method according to the invention that a gas generator, preferably an electrochemical gas generator, is used for providing and supplying the test gas in step b). Such gas generators can be used in a particularly flexible manner, by providing different concentrations of test gas. Gas generators are therefore preferred components of a test device for carrying out a method according to the invention.

According to an embodiment of a method according to the invention, it can be provided that a gas generation current and/or a gas generation duration of the gas generator is set on the basis of the test sequence defined in step a). Preferably, an electrochemical gas generator uses electricity to generate a test gas, for example, via electrolysis. By changing such a gas generation current and/or a gas generation duration, different test gas concentrations can thus be generated easily. By continuously changing the gas generation current over time, for example, it is particularly easy to enable a continuous change in such a test gas concentration over time.

A method according to an embodiment of the invention provides that in step d), when evaluating the sensor response determined in step c), an analysis is carried out as a first step, to find a basic pattern of the test sequence. In the sense of the invention, the basic pattern can be understood by whether, for example, a relative level of the various test gas concentrations provided is also represented by the determined sensor response. Whether, for example, gas flow pauses are represented in the determined sensor response can also be understood as part of finding a basic pattern of the test sequence. This first step is particularly simple and can provide a quick initial analysis of a functional status of a gas sensor.

In addition, a method according to an embodiment of the invention provides that in step d) at least one of the following further analyses is carried out when evaluating the sensor response determined in step c):

Temporal correlation between test sequence and sensor response

Strength of the sensor response

Rise behavior of the sensor response

Decay behavior of the sensor response

General requirement of the sensor response

Preferably several, more preferably, all, of these further analyses are carried out. This makes it possible to provide a qualitative and quantitative evaluation of the function of the gas sensor being checked.

According to a second aspect of the invention, the object is achieved by a test device for testing a gas sensor by applying a test gas. A test device according to the invention is characterized in that the test device is designed to carry out a method according to the first aspect of the invention. All the advantages which have been described in detail in relation to a method for testing a gas sensor according to the first aspect of the invention can thus also be provided by a test device according to the invention according to the second aspect of the invention, which is for carrying out a method according to the first aspect of the invention.

Furthermore, in a test device according to the invention, the test device preferably comprises a gas generator for generating the test gas and a control unit for controlling the gas generator and for determining a sensor response of the gas sensor. In this particularly preferred embodiment of a test device according to the invention, a control unit can be used, on the one hand, to operate a gas generator, by means of which a test gas is generated in accordance with the test sequence defined in step a) of a method according to the invention. The test gas generated is fed to the gas sensor to be checked. The control unit is also designed to determine the sensor response of the gas sensor. A continuous measurement of the gas sensor signals can be monitored by the control unit. A particularly compact embodiment of a test device according to the invention can be provided in this way due to the combined inclusion of the control unit and the gas generator.

According to a third aspect of the invention, the object is achieved by a system comprising a gas sensor and a test device for testing the gas sensor by applying a test gas. A system according to the invention is characterized in that the test device is designed according to the second aspect of the invention. A test device according to the second aspect of the invention is designed to carry out a method according to the first aspect of the invention. All of the advantages which have been described in detail in relation to a method according to the first aspect of the invention and in relation to a test device according to the second aspect of the invention can thus also be provided by a system according to the third aspect of the invention, which is a test device according to the second aspect of the invention or which carries out a method according to the first aspect of the invention.

Further measures for improving the invention result from the following description of exemplary embodiments of the invention, which are shown in the figures. All of the features and advantages arising from the claims, the description and the drawings, including structural details and spatial arrangements, can be essential to the invention both individually and in the various combinations. Elements with the same function and mode of operation are provided with the same reference symbols in the drawings:

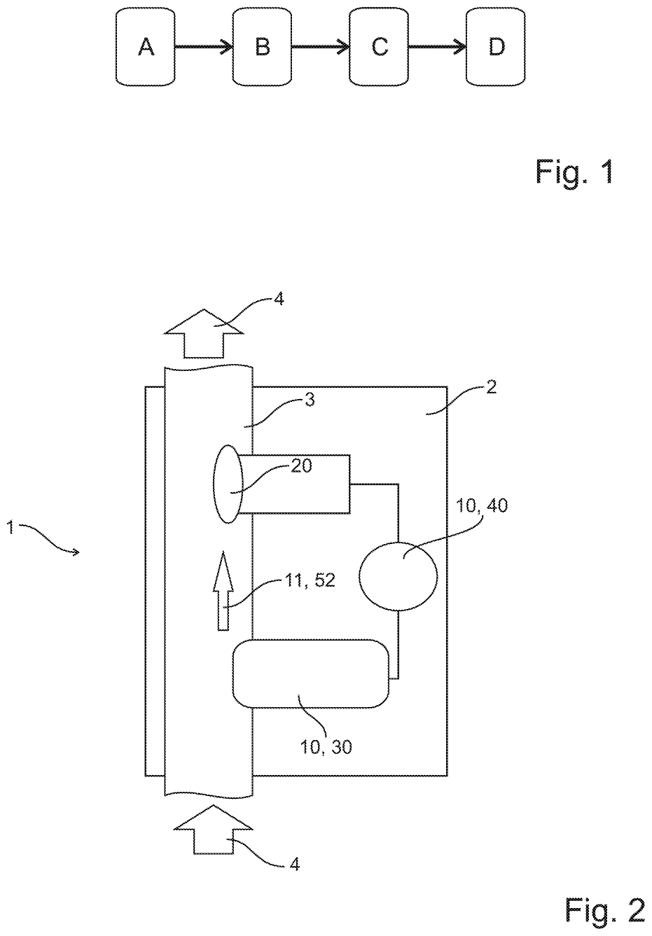

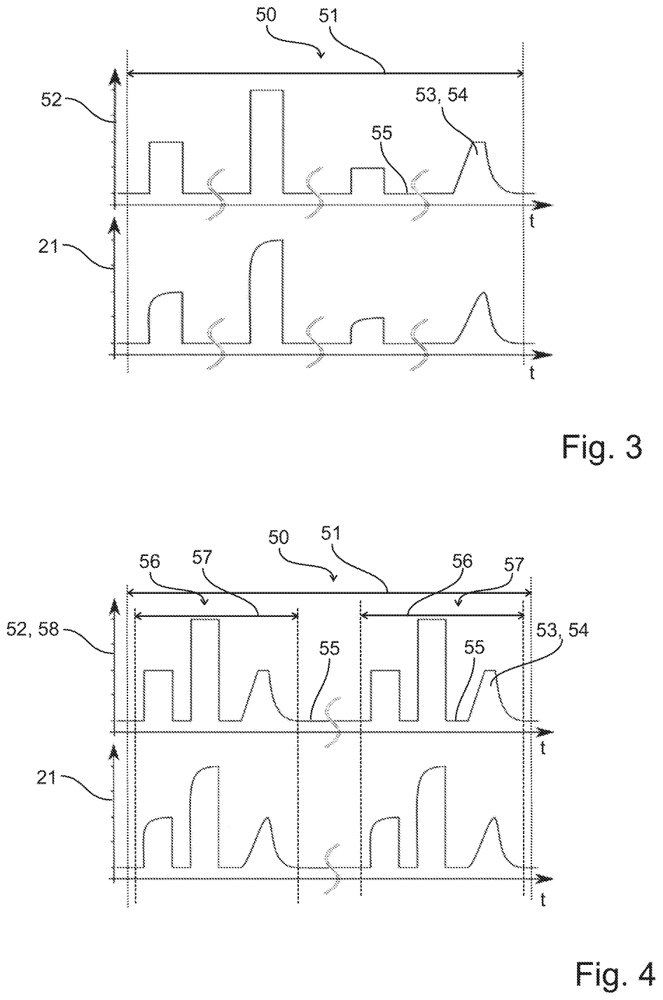

FIG. 1 shows a method according to an embodiment of the invention, which can be carried out by a system 1 according to the invention, as shown in FIG. 2. Test sequences 50 as shown in FIGS. 3 and 4 can be used. FIGS. 1 to 4 are therefore described together below, with the details of the individual figures being dealt with separately in each case.

A method according to an embodiment of the invention is shown in FIG. 1, steps a), b), c) and d) each being designated with capital letters. Such a method according to the invention can be carried out, for example, by a system 1 according to the invention, as shown in FIG. 2. Such a system 1 has a gas sensor 20 and a test device 10. The gas sensor 20 and the test device 10 can be arranged in a common housing 2, as a result of which a particularly compact embodiment of a system 1 can be provided. A configuration of the gas sensor 20 and the test device 10 can preferably be provided, each with its own housing element, which can preferably be arranged or attached to one another to form a common housing 2. In the illustrated embodiment of a system 1, a gas volume 3 is arranged in the housing 2 and is connected to ambient air 4. This connection between the gas volume 3 and the ambient air 4 can be provided by diffusion, or else by a suitable pump unit. The gas sensor 20 is arranged in the gas volume 3, as a result of which harmful gases, to which the gas sensor 20 is sensitive, can be detected in the ambient air 4. In this embodiment of a system 1, a gas generator 30 of the test device 10 is arranged upstream from the gas sensor 20. Controlled by a control unit 40, this gas generator 30, which can preferably be designed as an electrochemical gas generator 30, can generate a test gas 11 which is emitted as a test flow 52 into the ambient air 4 and is thus fed to the gas sensor 20. This is controlled by a control unit 40 of the test device 10, which is also designed to tap a sensor response 21, not shown in FIG. 2.

According to an embodiment of a method according to the invention, a test sequence 50 is defined in a first step a). Possible test sequences 50 are shown by way of example in FIGS. 3 and 4. Preferably, the defined test sequences 50 have a test duration 51, a test flow 52 of test gas 11 having at least two different test gas concentrations 53 during the test duration 51. To increase the clarity of FIGS. 3 and 4, only one of the test gas concentrations 53 is provided with reference numerals. The individual different test gas concentrations 53 can be separated from one another by gas flow pauses 55, as a result of which, the overall measurement accuracy when determining a functional status of the gas sensor 20 can be increased.

FIG. 3 shows four different test gas concentrations 53, which are each separated from one another by test flow pauses 55. One of the test gas concentrations 53 is also designed as a variable test gas concentration profile 54, in which, as shown, the test gas concentration 53 preferably changes continuously over time t. As shown, this can be trapezoidal, for example, at least in sections. Other shapes are also conceivable as the test gas concentration profile 54, including, but not limited to, a triangle, a ramp, steps and a sine wave.

In addition to individual test gas concentrations 53, as shown in FIG. 4, the test gas sequences 50, which are determined, for example, by the control unit 40, can also have at least two test sequence sections 56. These test sequence sections 56 are essentially sequences of test gas concentrations 53, which each form a unit in the entire test sequence 50. These can also be separated from one another by gas flow breaks 55. Preferably, as shown in FIG. 4, these test sequence sections 56 can be of identical design in order to further increase a measuring accuracy when determining the functional status of the gas sensor 20.

In the next step b) of a method according to an embodiment of the invention, a test gas 11 is provided based on the test sequences 50 defined in step a) and supplied to the gas sensor 20. This can be done, for example, by an electrochemical gas generator 30. The gas generation current and/or a gas generation duration of the gas generator 30 is controlled in accordance with the defined test sequence 50. The test gas 11 or the test flow 52 is released by the gas generator 30 and fed to the gas sensor 20. 3 and 4 each show a sensor response 21 to the respectively transmitted test flow 52 of test gas 11. It is clearly visible that the different profiles at test gas concentrations 53 also cause different forms of sensor response 21.

This is used in the next step d) of a method according to an embodiment of the invention, by evaluating the sensor response 21 determined in step c), in order to make an evaluation of the functional status of the gas sensor 20 and to complete the test of the gas sensor 20. In a first step of the evaluation, it is possible, to try to find basic patterns of the test sequences 50 used, for example a relative level of the sensor responses 21 in relation to the test gas concentrations 53 used or the presence of areas corresponding to the gas flow pauses 55, in the sensor response 21 of the gas sensor 20. Further analysis steps can also be carried out in order to determine, for example, temporal correlations between the test sequence 50 and the sensor response 21, a strength, a rising behavior or a decay behavior of the sensor response 21 or very generally a form of the sensor response 21.

In summary, testing the accuracy of a gas sensor 20 can be improved by a method according to the invention, a system 1 according to the invention or a test device 10 according to the invention. According to embodiments of the invention, the test gas 11 is fed to the gas sensor 20 in a test flow 52, with a test gas concentration 53 changing during a test period 51. The test duration 51 and test gas concentration 53 are combined in a test sequence 50. By analyzing the sensor response 21, resulting from the test sequence 50 used, qualitative and/or quantitative determinations of the functional status of the gas sensor 20 can be made.

### REFERENCE SIGN LIST

1 System
2 Casing
3 Gas volumes
4 Ambient air
10 Test device
11 Test gas
20 Gas sensor
21 Sensor response
30 Gas generator
40 Control unit
50 Test sequence
51 Test duration
52 Test flow
53 Test gas concentration
54 Test gas concentration profile
55 Gas flow break
56 Test sequence section
57 Test section duration
58 Test section flow
t Time

1. A method for testing a gas sensor by exposure to a test gas of a test device,
comprising the following steps:
a) determining a test sequence, the test sequence comprising at least one test duration and a test flow of the test gas during the test duration, the test flow also covering the test duration has at least two different test gas concentrations,
b) providing and supplying the test gas to the gas sensor on the basis of the test sequence defined in step a),
c) determining a sensor response of the gas sensor to the test flow of the test gas fed in step b), and
d) evaluating the sensor response determined in step c) to complete the test of the gas sensor.

2. The method according to claim 1, wherein
in step a) further comprises determining at least one of the at least two different test gas concentrations with a time-variable test gas concentration profile.

3. The method according to claim 2, wherein
the test gas concentration profile defined in step a) has at least one form selected from the group consisting of a triangle, a ramp, a rectangle, steps, a trapezoid, a sine wave, and combinations thereof.

4. The method according to claim 1, wherein
step a) further comprises determining a test sequence with at least one interruption of the flow of the test gas.

5. The method of claim 1,
wherein step a) further comprises defining the test sequence with two or more test sequence sections, the test sequence sections being arranged one after the other with respect to the test duration and in each case at least one test section duration and one test section flow of the test gas during the test section duration, the test section flow also having at least two different test gas concentrations over the test section duration.

6. The method of claim 5, wherein
step a) further comprises defining the two or more test sequence sections separately from one another by gas flow interruptions.

7. The method of claim 5, wherein
step a) further comprises defining the two or more test sequence sections identically.

8. The method of claim 1, further comprising supplying the test gas in step b) using a gas generator.

9. The method of claim 8, further comprising setting a gas generation current and/or a gas generation duration of the gas generator on the basis of the test sequence defined in step a).

10. The method according to claim 1, wherein
step d) further comprises carrying out an analysis to find a basic pattern of the test sequence as a first step in evaluating the sensor response determined in step c).

11. The method of claim 10, wherein step d) further comprises, as a subsequent step when evaluating the sensor response determined in step c), carrying out at least one further analysis selected from the group consisting of:
a temporal correlation between test sequence and sensor response;
a strength of the sensor response;
a rise behavior of the sensor response;
a decay behavior of the sensor response;
a general form of sensor response; and combinations thereof.

12. A test device for testing a gas sensor by applying a test gas, comprising
a test device designed to carry out the method of claim 1.

13. The test device of claim 12, further comprising
a test device comprising a gas generator for generating a test gas and a control unit for controlling the gas generator and for determining a sensor response of the gas sensor.

14. A system comprising a gas sensor and a test device for testing the gas sensor by applying a test gas wherein
the test device is designed according to claim 12.

15. The method of claim 8, wherein the gas generator is an electrochemical gas generator.
